(19) United States
(12) Patent Application Publication
(10) Pub. No.: US 2017/0135425 A1
(43) Pub. Date: May 18, 2017

(54) DISPOSABLE GLOVE WITH INTEGRATED FUNCTIONAL COATING LAYER AND METHOD OF MAKING THE SAME

(71) Applicant: Gang Hao, Corona, CA (US)

(72) Inventor: Gang Hao, Corona, CA (US)

(21) Appl. No.: 15/233,890

(22) Filed: Aug. 10, 2016

Related U.S. Application Data
(60) Provisional application No. 62/256,724, filed on Nov. 18, 2015.

(51) Int. Cl. A41D 19/00; A61B 42/10; A41D 19/04

(52) U.S. Cl. CPC A41D 19/0096; A41D 19/0068; A41D 19/0006; A41D 19/04; A61B 42/10; A41D 2500/54

(57) ABSTRACT

According to an embodiment of the present disclosure, a disposable glove may include a glove body having a shape of a hand; and a functional coating layer on a portion of the glove body and integrated with the glove body. According to another embodiment of the present disclosure, a method for manufacturing a disposable glove includes: coating a functional material formulation on a portion of a hand mold; drying the functional material formulation partially or completely to form a functional coating layer on the hand mold; coating the hand mold having the functional coating layer thereon with a glove body formulation; drying the glove body formulation to form a dried glove body; and removing the dried glove body from the hand mold so that the glove body is flipped inside out to form the disposable glove with the functional coating layer integrated with the glove body.

### CROSS-REFERENCE TO RELATED PATENT APPLICATION

This application claims priority to and the benefit of U.S. Provisional Patent Application No. 62/256,724, filed in the United States Patent and Trademark Office on Nov. 18, 2015, the entire content of which is incorporated herein by reference.

### FIELD OF THE INVENTION

The following description relates generally to disposable gloves and method of making the same. In particular, it relates to disposable gloves with integrated functional coating layer on a portion of the gloves, and method of making the same.

### BACKGROUND

Disposable gloves have been widely utilized in medical, industrial, clean room, food and other applications. The disposable gloves are typically made of one major polymeric material (plus small amount of additives in some cases) and are referred to according to the major material as nitrile gloves, vinyl gloves, natural rubber gloves, neoprene gloves, isoprene gloves, polyethylene (PE) gloves, etc.

Except for PE gloves, which are commonly made through a heat-stamp process, other types of disposable gloves are typically manufactured utilizing hand molds and a liquid containing the glove forming material (such as the glove forming material dissolved in a suitable solvent or in a molten state). The hand mold is first dipped into a tank containing the glove forming materials, followed by processes such as drying and peeling off the formed gloves from the hand mold. The process is an on-line continuous process which produces a large number of gloves in a short amount of time.

Due to restrictions in cost and the usage of the on-line production process, disposable gloves made of only one material component may have limitations inherent in the material utilized and imparted by the manufacturing process.

For example, related art disposable gloves that are manufactured with smooth surfaces do not possess good tactile sense and non-slip qualities. These gloves become slippery when in use, more so when in contact with water or fluid. Taking vinyl gloves as an example, polyvinyl chloride (PVC) based gloves (i.e., vinyl gloves) have been widely utilized because of the low cost of PVC material and the good processability of the material. However, these vinyl gloves can be slippery when handling wet subjects and therefore not suitable for such situations.

Polyethylene gloves are typically utilized for food handling. Due to the low melting point of polyethylene, these gloves are not suitable for handling hot food.

Latex gloves are also widely utilized. However, people may have allergic reaction to the latex material. Similarly, nitrile based gloves can be manufactured to be very thin, which provides good flexibility and cost-savings, but the fingertips of the gloves tend to break or puncture easily.

While non-disposable gloves, such as protective gloves made of fabric materials or with greater thickness than disposable gloves, may face similar issues, due to the much higher price these gloves are sold at, the issues may be mitigated by the more labor intensive process such as stitching or gluing of an additional functional portion, or simply by increasing the thickness of the whole glove. However, due to the lower price of disposable gloves, there is still a need to improve the performance of the disposable gloves without much increase in the cost and through a process that is compatible with the on-line production process.

### SUMMARY

Aspects according to one or more embodiments of the present invention are directed toward disposable gloves integrated with a functional coating layer and method of making the same.

Additional aspects will be set forth in part in the description which follows, and, in part, will be apparent from the description, or may be learned by practice of the presented embodiments.

According to an embodiment of the present disclosure, a disposable glove may include a glove body having a shape of a hand; and a functional coating layer on a portion of the glove body and integrated with the glove body.

The functional coating layer may be only on a location of the glove body selected from a thumbtip, an index fingertip, one or more fingertips, a palm, back of a hand, and a combination thereof.

The glove body may have a thickness of about 1.5 mil to about 5 mil, and the functional coating layer may have a thickness of about 1.5 mil to about 7 mil.

The functional coating layer may cover about 0.5% to about 80% of a total surface area of the glove body.

The glove body may include a first material selected from the group consisting of polyvinyl chloride, latex, nitrile, polyisoprene, and polyethylene.

The functional coating layer may include a second material, the second material being the same as or different from the first material.

The glove body may include polyvinyl chloride and the functional coating layer may include nitrile, latex, and/or polyisoprene.

The glove body may include nitrile and the functional coating layer may include nitrile.

The glove body may include latex and the functional coating layer may include nitrile, polyethylene, polyisoprene, and/or polyvinyl chloride.

The glove body may include polyethylene and the functional coating layer may include nitrile and/or latex.

The functional coating layer may include silicone and is located on a thumbtip, an index fingertip or both.

According to an embodiment of the present disclosure, a method for manufacturing a disposable glove includes: coating a functional material formulation on a portion of a hand mold; drying the functional material formulation partially or completely to form a functional coating layer on the hand mold; coating the hand mold having the functional coating layer thereon with a glove body formulation; drying the glove body formulation to form a dried glove body; and removing the dried glove body from the hand mold so that the glove body is flipped inside out to form the disposable glove with the functional coating layer integrated with the glove body.

The coating of the functional material formulation may be only on a location of the hand mold selected from a thumbtip, an index fingertip, one or more fingertips, a palm, back of a hand, and a combination thereof.

The functional coating layer may cover about 0.5% to about 80% of a total surface area of the glove body.

The glove body formulation may include a first material selected from the group consisting of polyvinyl chloride, latex, nitrile, polyisoprene, and polyethylene.

The functional material formulation may include a second material, the second material being the same as or different from the first material.

The glove body formulation may include polyvinyl chloride and the functional material formulation may include nitrile, latex, and/or polyisoprene.

The glove body formulation may include nitrile and the functional material formulation may include nitrile.

The glove body formulation may include latex and the functional material formulation may include nitrile, polyethylene, and/or polyvinyl chloride.

The glove body formulation may include polyethylene and the functional material formulation may include nitrile and/or latex.

According to an embodiment of the present disclosure, a disposable glove may include one or more textured portions disposed on one or more fingertips and a thumbtip. The textured portions on this glove are intended to provide the enhanced grip for those persons who require a sure grip at their fingertips, particularly surgeons and dental professionals. In one embodiment, the textured portions are located on the thumbtip, index fingertip and middle fingertip. In another embodiment, the glove may be made by, but not limited to PE, vinyl materials, nitrile rubber, latex, etc. In a further embodiment, the textured portions may be made by, but not limited to, nitrile, latex, polyisoprene or other non-slippery or other functional material (such as a puncture protection material).

In another embodiment, a method for producing a disposal glove with one or more functional portions may include: preparing a hand mold coated with functional materials on set or predetermined portions of the hand mold; preparing a glove made by base materials; coating the functional materials onto an inner surface of the glove by inserting the glove into the hand mold; and flipping the functional materials from the inner surface of the glove to an outer surface of the glove.

In one embodiment, the glove may be made by, but not limited to, PE, vinyl materials, nitrile rubber, latex, etc. In another embodiment, the functional materials may be, but not limited to, nitrile, latex, polyisoprene or the like, or other functional materials (like puncture protection material).

In an exemplary embodiment, the set or predetermined portions to coat the functional materials on the mold are selected from the group consisting of thumbtip, index fingertip, middle fingertip, and combinations thereof.

### BRIEF DESCRIPTION OF THE DRAWINGS

FIG. 1 is a schematic illustration of a disposable glove according to one embodiment of the present invention.

FIG. 2 is a sectional view of a finger of a disposable glove according to one embodiment of the present invention.

FIG. 3 is a flow chart illustrating a method of manufacturing a disposable glove according to an embodiment of the present disclosure.

FIG. 4 is a schematic illustration of a method of coating a functional material formulation on a portion of a hand mold.

FIG. 5 is a schematic illustration of a disposable glove with a textured portion according to an embodiment of the present disclosure.

FIG. 6 schematically illustrates a hand mold with functional coating layer coated thereon.

### DETAILED DESCRIPTION

Reference will now be made in more detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments of the present disclosure are merely described below, by referring to the figures, to explain aspects of the present description.

As used herein, the term “and/or” includes any and all combinations of one or more of the associated listed items. Expressions such as “at least one of,” when preceding a list of elements, modify the entire list of elements and do not modify the individual elements of the list.

As the inventive concept allows for various changes and numerous embodiments, particular embodiments will be illustrated in the drawings and described in more detail in the written description. Effects, features, and a method of achieving the inventive concept will be obvious by referring to exemplary embodiments of the inventive concept with reference to the accompanying drawings. The inventive concept may, however, be embodied in many different forms and should not be construed as being limited to the embodiments set forth herein.

In the embodiments described in the present specification, an expression utilized in the singular encompasses the expression of the plural, unless it has a clearly different meaning in the context. Also, it is to be understood that the terms such as “including,” “having,” and/or “comprising” are intended to indicate the presence of the stated features or components, and are not intended to preclude the presence or addition of one or more other features or components.

It will be understood that when a layer, region, or component is referred to as being “on” or “onto” another layer, region, or component, it may be directly or indirectly formed on the other layer, region, or component. That is, for example, intervening layer(s), region(s), or component(s) may be present.

Sizes of components in the drawings may be exaggerated for convenience of explanation. In other words, since sizes and thicknesses of components in the drawings are arbitrarily illustrated for convenience of explanation, the following embodiments of the present disclosure are not limited thereto.

The detailed description set forth below is intended as a description of the presently exemplary device provided in accordance with aspects of the present invention and is not intended to represent the only forms in which the present invention may be prepared or utilized. It is to be understood, rather, that the same or equivalent functions and components may be accomplished by different embodiments that are also intended to be encompassed within the spirit and scope of the invention.

Unless defined otherwise, all technical and scientific terms used herein have the same meaning as commonly understood to one of ordinary skill in the art to which this invention belongs.

Although any methods, devices and materials similar or equivalent to those described can be used in the practice or testing of the invention, the exemplary methods, devices and materials are now described.

According to an embodiment of the present disclosure, a disposable glove may include a glove body having a shape of a hand; and a functional coating layer on a portion of the glove body and integrated with the glove body.

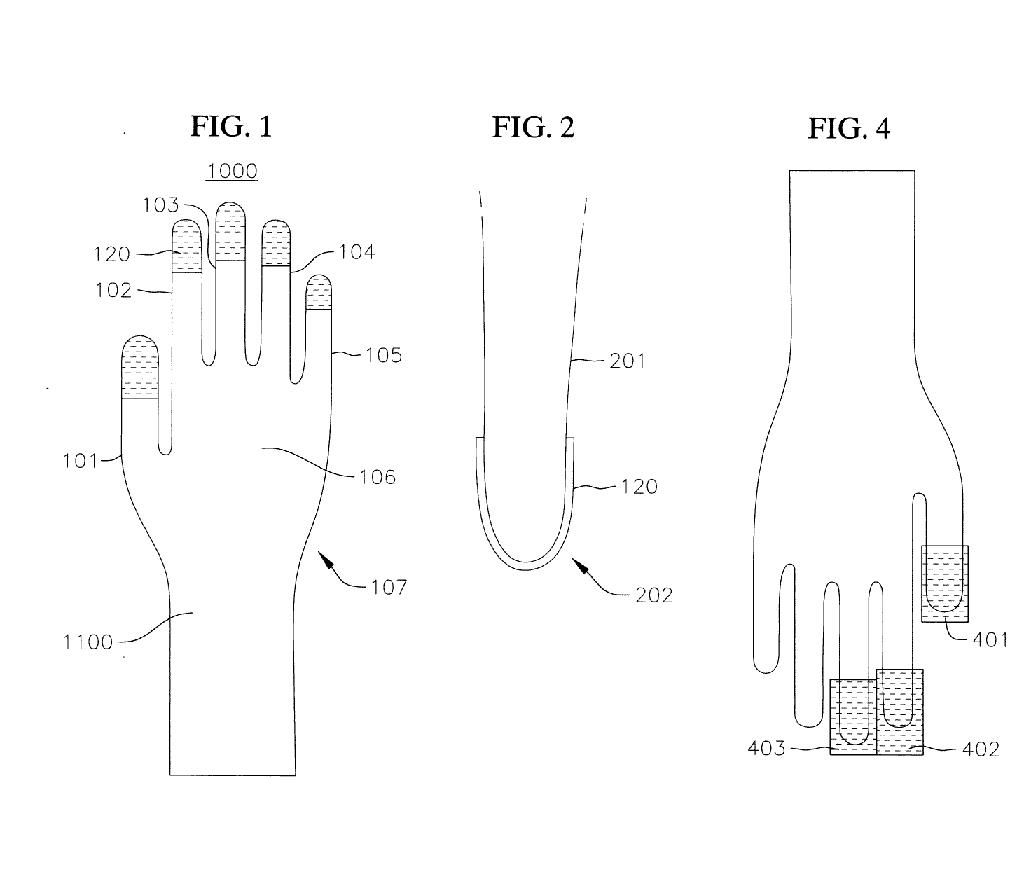

FIG. 1 is a schematic illustration of a disposable glove according to one embodiment of the present invention. Referring to FIG. 1, the disposable glove 1000 includes a glove body 1100 having the shape of a hand. The glove body 1100 includes five fingers (a thumb 101, an index finger 102, a middle finger 103, a ring finger 104, and a little finger 105), a palm 106, and a back side 107. The disposable glove 1000 also includes a functional coating layer 120 on a portion of the glove body 1100 and integrated with the glove body 1100. Here, the term “integrated” indicates that the functional coating layer is not separable from the glove body through normal usage of the disposable glove, and refers to the attachment of the functional coating layer to the glove body through molecular bonding between the material for forming the glove body and the material for forming the functional coating layer, without an additional adhesive layer or other adhesion mechanisms between the glove body and the functional coating layer.

The functional coating layer may not be on the whole glove body and may be only on one or more portions of the glove body. For example, the functional coating layer may be on a location of the glove body selected from a thumbtip, an index fingertip, one or more fingertips, one or more of the five fingers of a hand, a palm, back of a hand, and a combination thereof. In one embodiment, the functional coating layer is only on the thumbtip of the glove body. In another embodiment, the functional coating layer is only on the index fingertip of the glove body. In another embodiment, the functional coating layer is only on both the thumbtip and the index fingertip of the glove body. In another embodiment, the functional coating layer is on five fingertips of the glove body. In one embodiment, the functional coating layer is only on the front side of a fingertip (i.e., the pad side) and not on the back side of the fingertip (i.e., the nail side). In another embodiment, the functional coating layer surrounds the fingertip and is on both the front side and back side of a fingertip. According to another embodiment, the functional coating layer is on the five fingers of a hand, but not on the palm or back of the hand.

FIG. 2 is a sectional view of a finger of a disposable glove according to one embodiment of the present invention. Referring to FIG. 2, the functional coating layer 120 surrounds the fingertip portion 202 of the finger 201.

The functional coating layer on the “fingertip” and “thumbtip” may cover about 10% to about 100% of the surface area of the respective fingertip, for example, about 30% to about 90% of the surface area of the fingertip. Here, the term “fingertip” or “thumbtip” refers to the portion of the respective finger that includes from the end of the finger to the joint closest to the end of the finger, and the term “fingertip” may refer to this portion of the finger of any or all of the five fingers of a hand. For example, a thumbtip may also be referred to as a fingertip.

Due to the cost restriction and the processing conductions, the disposable gloves are usually significantly thinner than other kinds of gloves, such as protective gloves or multiple-use gloves. In one embodiment, the glove body may have a thickness of about 1 mil to about 15 mil, for example, about 1.5 mil to about 3 mil.

In one embodiment, the functional coating layer may have a thickness of about 1.5 mil to about 20 mil, for example, about 1.5 mil to about 6 mil.

The functional coating layer may not cover the total surface area of the glove body, and may only be applied to specific areas of interest, as disclosed above. In one embodiment, the functional coating layer may cover about 0.5% to about 80% of a total surface area of the glove body. For example, the functional coating layer may cover about 0.5% to about 50% or about 2% to about 10% of the total surface area of the glove body. When the functional coating layer is applied to the specific areas of interest at these coverage ranges, improvement in performance and cost savings may be realized.

The glove body may include a first material selected from the group consisting of polyvinyl chloride (PVC), latex, nitrile, polyisoprene, and polyethylene (PE). That is, the disposable glove may be a vinyl-based glove, a latex-based glove, a nitrile-based glove, a polyisoprene-based glove (also referred to as an isoprene-based glove) or a PE-based glove according to the material utilized to form the glove body. The glove body is made of a polymeric material and is non-porous.

The functional coating layer may include a second material, the second material may be the same as or different from the first material included in the glove body. The second material may be any suitable material that provides the desired properties such as non-slippery surface, non-allergic reaction, enhanced mechanical strength, tear and puncture resistance, and/or heat resistance.

In one embodiment, the desired property is non-slippery surface. A suitable functional coating layer for non-slippery surface may include nitrile, latex, and/or polyisoprene.

For example, the glove body may include polyvinyl chloride (e.g., vinyl) and the functional coating layer may include nitrile, latex, and/or polyisoprene coated on a portion of the glove body where such a property is desired, such as fingertips and thumbtip. For example, the functional coating layer may be coated on all five fingertips, or only on the thumbtip and the index fingertip. The functional coating layer may cover about 2% to about 10% of the total surface area of the glove body. Related art vinyl-based disposable gloves are not suitable for handling wet subjects as the gloves become slippery when in contact with liquids. The vinyl-based gloves including the functional coating layer according to embodiments of the present invention may overcome the slippery issues due to the non-slippery functional coating layer.

In another embodiment, the desired property is enhanced tear and puncture resistance. A suitable functional coating layer for enhanced tear and puncture resistance may include a same material as that of the glove body, but locally strengthen the glove with a greater thickness.

For example, the glove body may include nitrile and the functional coating layer may include nitrile. The glove body may have a thickness of about 1.5 mil to about 5 mil except where the functional coating layer is located. The four fingertips and the thumbtip of the disposable glove may include the functional coating layer, and the thickness of the functional coating layer may be about 1.5 mil to about 7 mil. With the enhanced thickness at the fingertips and thumbtip, the resistance to tear and puncture may be greatly improved without much increase in the cost of making the gloves, since the functional coating layer is only applied to the fingertips and thumbtip of the disposable glove.

In another embodiment, the desired property is non-allergic reaction. A suitable functional coating layer for non-allergic reaction may include a non-latex based material, such as nitrile, vinyl, polyisoprene, and/or PE.

For example, the glove body may include latex and the functional coating layer may include nitrile, polyethylene, and/or polyvinyl chloride. The functional coating layer may be applied to locations of the glove body that may come into contact with a patient. In one embodiment, each of the five fingers of the disposable glove is coated with the functional coating layer. In another embodiment, only the fingertip of each of the five fingers of the disposable glove is coated with the functional coating layer. When a healthcare professional, such as a physician and/or a dentist, uses the latex gloves including the functional coating layer to touch a patient, because the portion of the disposable glove contacting the patient is coated with the non-allergic material, allergic reaction in the patient may be avoided.

In another embodiment, the desired property is heat resistance. A suitable functional coating layer for heat resistance may include a high density polyethylene (HDPE), vinyl, etc.

For example, the glove body may include polyethylene of lower melting point (such as low density polyethylene) and the functional coating layer may include high density polyethylene (HDPE) and/or vinyl. When the functional coating layer is applied to portions of the disposable glove that will come in contact with hot items, such as the fingertips, the PE based disposable glove may be able to handle the hot items without any melting issues.

In one embodiment, the functional coating layer may include silicone and is located on a thumbtip, an index fingertip or both. When a disposable glove includes such a functional coating layer, the disposable glove may be utilized to operate touch sensitive devices, such as a smart phone, a tablet, etc.

The disposable gloves according to one or more embodiments of the present disclosure may be utilized as surgical gloves, medical exam gloves, food handling gloves, and/or general purpose gloves.

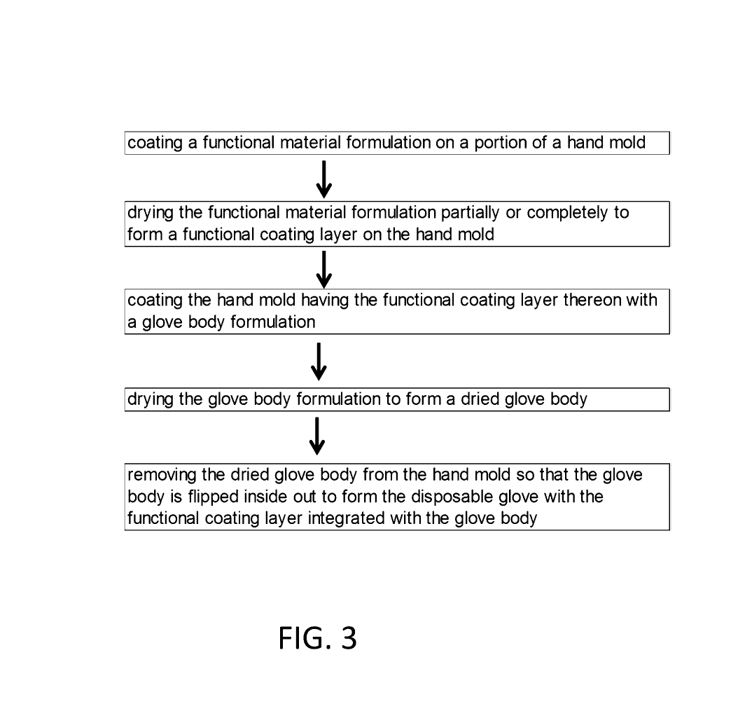

FIG. 3 is a flow chart illustrating a method of manufacturing a disposable glove according to an embodiment of the present disclosure. Referring to FIG. 3, a method for manufacturing a disposable glove includes: coating a functional material formulation on a portion of a hand mold; drying the functional material formulation partially or completely to form a functional coating layer on the hand mold; coating the hand mold having the functional coating layer thereon with a glove body formulation; drying the glove body formulation to form a dried glove body; and removing the dried glove body from the hand mold so that the glove body is flipped inside out to form the disposable glove with the functional coating layer integrated with the glove body. The functional coating layer is on the outside of the glove.

The functional material formulation may be a liquid including a second material dissolved or dispersed in a suitable solvent, or the second material in the molten state. In one embodiment, the coating of the functional material formulation on a portion of a hand mold may include dipping a portion of the hand mold, for example, one or more fingers of the hand mold into the functional material formulation and lifting the hand mold out of the functional material formulation.

FIG. 4 is a schematic illustration of a method of coating a functional material formulation on a portion of a hand mold. Referring to FIG. 4, the thumb of the hand mold is dipped into a container 401 containing a first functional material formulation; the index finger of the hand mold is dipped into a container 402 containing a second functional material formulation; and the middle finger of the hand mold is dipped into a container 403 containing a third functional material formulation. The first, second and third functional material formulations may be the same or different. Here, each of the containers 401, 402 and 403 may be arranged at a different height corresponding to the length of respective fingers such that the functional coating layer is coated at the same length for each of the fingers. In another embodiment, the liquid level in each of the containers 401, 402 and 403 may be arranged at a different height corresponding to the length of respective fingers such that the functional coating layer is coated at the same length for each of the fingers. In another embodiment, a single container is utilized and all fingers are dipped into the single container to have a functional coating layer with different length for each of the fingers.

The coating of the functional material formulation may be conducted in other suitable methods, such as spray coating, where the functional material formulation may be sprayed on the desired portions of the hand mold, such as the palm, the back side of the hand mold, and/or the fingertips.

The drying of the functional material formulation partially or completely to form a functional coating layer on the hand mold may be conducted utilizing any suitable methods, such as drying in a pre-heated oven, and/or drying with hot air. The functional material formulation may be dried completely, or partially such that no dripping occurs.

After the functional material formulation is dried to the desired degree, the coating of the hand mold having the functional coating layer thereon with a glove body formulation may be conducted utilizing any suitable apparatus, for example, utilizing the related art production equipment and process for manufacturing disposable gloves. The glove body formulation may be a liquid including a first material dissolved or dispersed in a suitable solvent. Here, the glove body formulation may be the same as related art formulations utilized for forming a disposable glove. In one embodiment, the coating of the hand mold having the functional coating layer thereon with a glove body formulation may include dipping the hand mold into the glove body formulation and lifting the hand mold out of the glove body formulation.

The drying of the glove body formulation to form a dried glove body may be conducted utilizing any suitable methods, such as drying in a pre-heated oven, and/or drying with hot air. Through the drying of the glove body formulation, the formed glove body is integrally bonded with the functional coating layer. In case where the functional coating layer is only partially dried prior to the coating of the hand mold having the functional coating layer thereon with a glove body formulation, the functional coating layer also becomes completely dried in this act.

In the removing of the dried glove body from the hand mold, the glove body is flipped inside out to form the disposable glove with the functional coating layer integrated with the glove body. Here, after the glove body is flipped inside out, the functional coating layer is on the outside of the glove. As such, a disposable glove with integrated functional coating layer has been manufactured.

The coating of the functional material formulation may be only on a location of the hand mold selected from a thumbtip, an index fingertip, one or more fingertips, a palm, back of a hand, and a combination thereof.

The functional coating layer may cover about 0.5% to about 80% of a total surface area of the glove body.

The glove body formulation may include a first material selected from the group consisting of polyvinyl chloride, latex, nitrile, polyisoprene, and polyethylene. Here, the first material is a polymeric material.

The functional material formulation may include a second material, the second material being the same as or different from the first material. The second material is a polymeric material.

The glove body formulation may include polyvinyl chloride, and the functional material formulation may include nitrile, latex, and/or polyisoprene.

The glove body formulation may include nitrile, and the functional material formulation may include nitrile.

The glove body formulation may include latex, and the functional material formulation may include nitrile, polyethylene, and/or polyvinyl chloride.

The glove body formulation may include polyethylene, and the functional material formulation may include nitrile and/or latex.

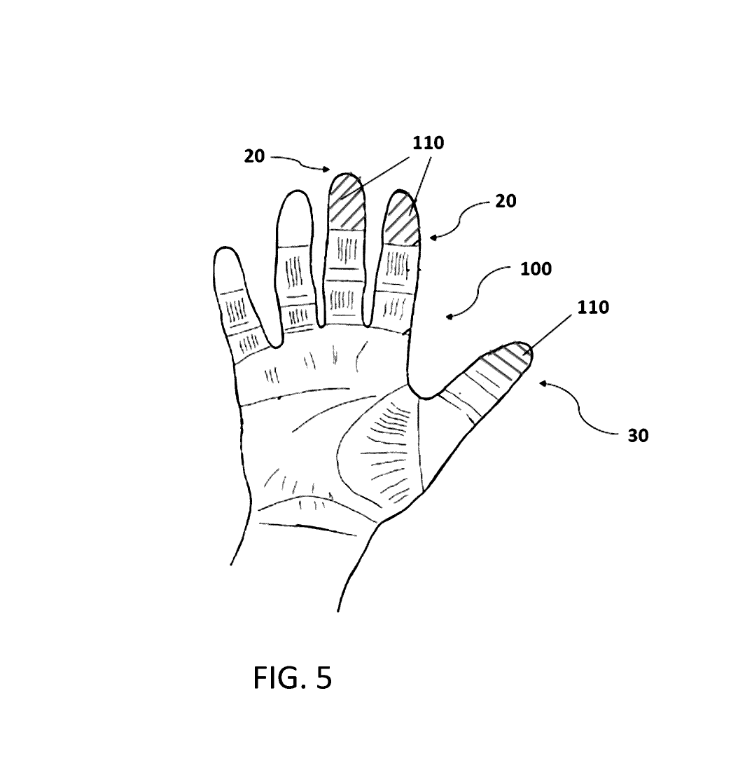

FIG. 5 is a schematic illustration of a disposable glove with a textured portion according to an embodiment of the present disclosure. Referring to FIG. 5, a disposable glove 100 may include a textured portion 110 disposed on one or more fingertips 20 and a thumbtip 30. The textured portions 110 on this glove 100 are intended to provide the enhanced grip for those persons who require a sure grip at their fingertips, particularly surgeons and dental professionals. In one embodiment, the textured portions 110 are located on the thumbtip 30, index and middle fingertips 20. In another embodiment, the glove 100 can be made by, but not limited to PE, vinyl materials, nitrile rubber, latex, etc. In a further embodiment, the textured portions 110 can be made by, but not limited to nitrile, latex, polyisoprene or other non-slippery material or other functional material (like puncture protection material).

It is noted that the vinyl material is known to be slippery, so when surgeons or dental professionals wearing the vinyl glove need to handle small instruments, it may be difficult for them to handle such a small object without the textured portions 110.

In another embodiment, a method for producing a disposable glove with one or more functional portions may include: preparing a hand mold coated with a functional material on one or more set or predetermined portions of the mold; preparing a glove made by base materials; coating the functional materials onto an inner surface of the glove by inserting the glove into the hand mold; and flipping the functional materials from the inner surface of the glove to an outer surface of the glove.

In one embodiment, the glove can be made by, but not limited to, PE, vinyl materials, nitrile rubber, latex, etc. In another embodiment, the functional materials can be, but not limited to, nitrile, latex, polyisoprene or the like, or other functional materials (like puncture protection material).

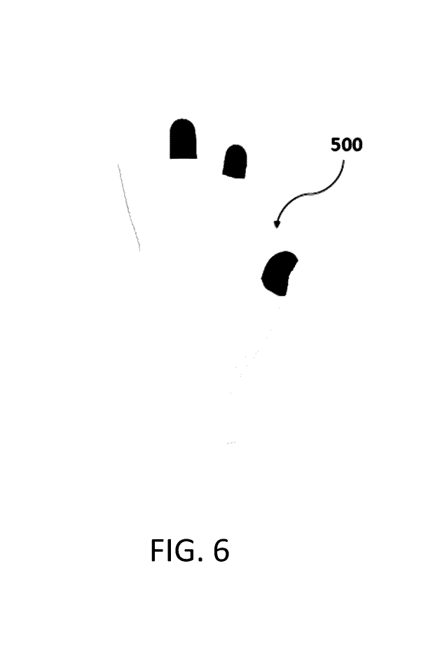

FIG. 6 schematically illustrates a hand mold with functional coating layer coated thereon. Referring to FIG. 6, in an exemplary embodiment, the set or predetermined portions to coat the functional materials on the mold 500 are thumb tip, and index and middle fingertips.

Compared with related art manufacturing processes of disposable glove with functional portions, the manufacturing method in the present invention is much more straightforward and easier, which can significantly save the labor and manufacturing costs.

Having described the invention by the description and illustrations above, it should be understood that these are exemplary of the invention and are not to be considered as limiting. Accordingly, the invention is not to be considered as limited by the foregoing description, but includes any equivalents. For example, while a disposable glove having five fingers has been described, embodiments of the present disclosure are not limited thereto, and the disposable glove may be in the shape of a mitten with a thumb and a section for the rest of the fingers.

The foregoing description of the preferred embodiments of the invention has been presented for purposes of illustration and description and is not intended to be exhaustive or to limit the claimed invention to the precise form disclosed. Those of skill in the art will readily appreciate that many modifications and variations to the claimed invention are possible in light of the above teaching. The embodiments were chosen and described in order to best explain the principles of the invention and its practical application to thereby enable others skilled in the art to best utilize the invention in various photocathode embodiments and with various modifications as are suited to the particular use contemplated. It is intended that the scope of the invention be defined exclusively by the following claims, and equivalents thereof.

We claim:

1. A disposable glove, comprising:
a glove body having a shape of a hand; and
a functional coating layer on a portion of the glove body and integrated with the glove body.

2. The disposable glove of claim 1, wherein the functional coating layer is only on a location of the glove body selected from a thumbtip, an index fingertip, one or more fingertips, a palm, back of a hand, and a combination thereof.

3. The disposable glove of claim 1, wherein the glove body has a thickness of about 1.0 mil to about 15 mil, and the functional coating layer has a thickness of about 0.5 mil to about 5 mil.

4. The disposable glove of claim 1, wherein the functional coating layer covers about 0.5% to about 80% of a total surface area of the glove body.

5. The disposable glove of claim 1, wherein the glove body comprises a first material selected from the group consisting of polyvinyl chloride, latex, nitrile, polyisoprene, and polyethylene.

6. The disposable glove of claim 5, wherein the functional coating layer comprises a second material, the second material being the same as or different from the first material.

7. The disposable glove of claim 1, wherein the glove body comprises polyvinyl chloride, and the functional coating layer comprises nitrile, latex, and/or polyisoprene.

8. The disposable glove of claim 1, wherein the glove body comprises nitrile, and the functional coating layer comprises nitrile.

9. The disposable glove of claim 1, wherein the glove body comprises latex, and the functional coating layer comprises nitrile, polyethylene, and/or polyvinyl chloride.

10. The disposable glove of claim 1, wherein the glove body comprises polyethylene, and the functional coating layer comprises nitrile and/or latex.

11. The disposable glove of claim 1, wherein the functional coating layer comprises silicone and is located on a thumbtip, an index fingertip or both.

12. A method for manufacturing a disposable glove, the method comprising:
coating a functional material formulation on a portion of a hand mold;
drying the functional material formulation partially or completely to form a functional coating layer on the hand mold;
coating the hand mold having the functional coating layer thereon with a glove body formulation;
drying the glove body formulation to form a dried glove body; and
removing the dried glove body from the hand mold to flip the glove body inside out to form the disposable glove with the functional coating layer integrated with the glove body.

13. The method of claim 12, wherein the coating of the functional material formulation is only on a location of the hand mold selected from a thumbtip, an index fingertip, one or more fingertips, a palm, back of a hand, and a combination thereof.

14. The method of claim 12, wherein the functional coating layer covers about 0.5% to about 80% of a total surface area of the glove body.

15. The method of claim 12, wherein the glove body formulation comprises a first material selected from the group consisting of polyvinyl chloride, latex, nitrile, polyisoprene, and polyethylene.

16. The method of claim 15, wherein the functional material formulation comprises a second material, the second material being the same as or different from the first material.

17. The method of claim 12, wherein the glove body formulation comprises polyvinyl chloride, and the functional material formulation comprises nitrile, latex, and/or polyisoprene.

18. The method of claim 12, wherein the glove body formulation comprises nitrile, and the functional material formulation comprises nitrile.

19. The method of claim 1, wherein the glove body formulation comprises latex, and the functional material formulation comprises nitrile, polyethylene, and/or polyvinyl chloride.

20. The method of claim 1, wherein the glove body formulation comprises polyethylene, and the functional material formulation comprises nitrile and/or latex.
